(19) United States
(12) Patent Application Publication
(10) Pub. No.: US 2009/0092668 A1
(43) Pub. Date: Apr. 9, 2009

(54) ACETAMINOPHEN FORMULATION FOR JOINT PAIN RELIEF

(76) Inventor: David FINKELSTEIN, Boca Raton, FL (US)

Correspondence Address: FROMMER LAWRENCE & HAUG, 745 FIFTH AVENUE- 10TH FL., NEW YORK, NY, 10151, US

(21) Appl. No.: 12/257,645

(22) Filed: Oct. 24, 2008

Related U.S. Application Data
(63) Continuation of application No. 11/399,578, filed on Apr. 6, 2006, now abandoned.

(51) Int. Cl. A61K 9/28; A61K 31/70; A61P 25/00

(52) U.S. Cl. 424/463; 514/62; 424/474

(57) ABSTRACT

The invention relates generally to a formulation which may comprise additional vitamins, minerals, herbs and supplements and methods for using the same for joint pain relief. The formulation may comprise supplements such as glucosamine, hyaluronic acid and methylsulfonylmethane (MSM) and acetaminophen for acute joint pain. The invention also encompasses methods for joint pain relief with the formulation described herein.

### INCORPORATION BY REFERENCE

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The invention relates generally to an acetaminophen formulation which may comprise additional vitamins, minerals, herbs and supplements and methods for using the same for joint pain relief.

### BACKGROUND OF THE INVENTION

Degradation of the structures in articular cartilage is a typical characteristic of all diseases resulting in chronic destruction of the joint structures. Examples of such disorders are rheumatoid arthritis, psoriatic arthritis, and osteoarthrosis. Also, acute inflammation of a joint is often accompanied by destruction of the cartilage, although in most cases this will not develop into the chronically destructive disease. It is not known which factors are crucial for the acutely inflamed joint to either proceed to healing or develop into the chronic process. Examples of diseases involving acute joint inflammation are yersinia arthritis, pyrophosphate arthritis, gout arthritis (arthritis urica), septic arthritis and various forms of arthritis of traumatic etiology. Among other factors potentially conducive to the destruction of articular cartilage may be mentioned, for instance, treatment with cortisone; this has been known for a long time to accelerate the degenerative process in osteoarthrosis.

Osteoarthritis and rheumatoid arthritis are representative of the diseases accompanied by the destruction of the cartilage matrix. It is thought that the destruction of the matrix in these diseases is triggered by mechanical stresses with aging in the case of osteoarthritis and by excess proliferation of the surface layer cells of the synovial membrane, pannus formation and inflammatory cell infiltration in the case of rheumatoid arthritis, and both phenomena are caused through the induction of proteases. Since the degradation of articular cartilage is progressed in the extracellular region at a neutral pH, it is said that a matrix metalloprotease (hereinafter referred to as “MMP” or “MMPs” when used as the general term) whose optimal pH is in the neutral range plays a leading role in the degradation.

No medical cure exists for osteoarthritis. The progressive degeneration of the joint due to osteoarthritis is irreversible. Present therapies are directed to palliative medical therapies to reduce inflammation and pain and surgical therapies to reconstruct an affected joint or, in severe cases, to replace the joint with an artificial, prosthetic joint.

Glucosamine is an essential intermediate in the biosynthetic pathway of proteoglycans, which are the primary building blocks of connective tissue and cartilage. Glucosamine compounds exhibit weak anti-inflammatory activity, but no analgesic activity. Glucosamine in combination with manganese and chondroitin, which is also a component of proteoglycans, is currently marketed as a nutritional supplement to enhance the repair and synthesis of connective tissue and cartilage (See U.S. Pat. Nos. 5,364,845; 5,587,363; 5,840,715). Glucosamine combined with ascorbic acid, tyrosine or phenylalanine, and calcium has been shown to accelerate wound healing (See U.S. Pat. Nos. 4,647,453; 4,772,591; and 5,679,344). Glucosamine has also been used to improve the solubility of NSAIDs by combining a glucosamine with a nonsteroidal anti-inflammatory drug in a 1:1 molar ratio to form a glucosamine salt or complex with the nonsteroidal anti-inflammatory drug, but the analgesic effect (whether sub-additive, additive or synergistic) has not been reported for these complexes (See U.S. Pat. Nos. 4,501,727; 5,604,206; and 6,069,172).

Hyaluronic acid is a naturally occurring glycosaminoglycan. Hyaluronic acid is ubiquitous in the organism, with the highest concentration found in soft connective tissue and joint fluid. It is a constituent of the intercellular matrix of connective tissue that exists in almost all vertebrates. It plays an important role in a number of physiological functions, including protection and lubrication of cells, maintenance of the structural integrity of tissues, transport of molecules and cells, cell migration, cell function and differentiation, and fluid retention and regulation. The clinical benefits of intra-articular hyaluronic acid in the horse are well published.

Hyaluronic acid is an important component of the intercellular matrix. Specifically, the highest levels are found in the eye and synovial fluid of joints. In joints, its primary role is that of lubrication, reducing pain, and inflammation. In arthritic joints hyaluronic acid is deficient. In regards to the joints, synovial fluid supplies nutrition to the articular cartilage and has incomparable functions as a lubricant and a shock absorber. It is clarified that its excellent viscoelastisity heavily owes to one of the main components, hyaluronic acid. Concentration and molecular weight analyses of hyaluronic acid demonstrated the concentration and molecular weight of hyaluronic acid in the synovial fluid from patients with arthritis such as osteoarthritis and chronic articular rheumatism generally tended to be lower than in normal synovial fluid, and the lower concentration and molecular weight of hyaluronic acid were closely associated with development of locomotor dysfunction and pain attributable to the weaker lubricating action and the weaker protecting action on the surface of the articular cartilage of synovial fluid.

Methylsulfonylmethane (MSM), a volatile component in the sulfur cycle, is another source of sulfur found in the human diet. Increases in serum sulfate may explain some of the therapeutic effects of MSM, DMSO, and glucosamine sulfate. Because MSM is naturally present in the body fluids and tissues of most if not all normal mammals, its mechanism of treating the physiological symptoms of stress appear to be less than that of a drug and more like a dietary supplement, such as is achieved with large dosages of vitamins. Although MSM has not yet been established to be a vitamin, at least a vitamin deficiency-type disease has not yet been shown to occur in patients with abnormally low MSM blood levels, it does have a vitamin-like moderating or normalizing activity for various body functions, as there appears to be a high correlation between abnormal physiological symptoms and low MSM blood levels in human beings. Whether this is due to the inability of such individuals to adequately store MSM from natural sources thereof, to inadequate amounts of MSM in the diet of those individuals or to the depletion of the MSM usually present in the body as a result of the abnormal condition, is not known. Whatever the reason, the oral ingestion of MSM in sufficient amounts will ultimately bring MSM levels to or above those usually present in healthy mammals and will ameliorate a variety of symptoms associated with stress.

Glucosamine is a component of all human tissue and is found in especially high concentrations in the cartilage. Chemically an aminomonosaccharide, glucosamine provides the building blocks for the O-linked and N-linked glycosaminoglycans comprising the matrix of the connective tissues in the body. The sulfate form is readily absorbed from the small intestine—over 90%. Of the absorbed glucosamine, 25% will be excreted in the urine, 65% excreted as exhaled carbon dioxide, and 10% remaining in the tissues. Once it is taken up into the chondrocytes of cartilage, glucosamine is incorporated into proteoglycans. There have been no reports of significant drug interactions of glucosamine with antibiotics or antidepressants.

In U.S. Pat. No. 4,559,329, one subject presented chronic arthritis with painful involvement of the lower trunk. Over the years she had evaluated most new antiarthritic, analgesic drugs with disappointing results. She included MSM at ½ tsp. daily in her diet and found almost total pain relief by the end of the second week. After ingesting MSM daily at ¼-½ tsp. for about 16 months, the subject enjoyed a substantially pain-free life.

Although MSM is found as a natural constituent of foodstuffs, like vitamin D the principal supply in vertebrates is believed to be synthesized by the body using dimethyl sulfide or one of its naturally occurring precursor salts as commonly found in meat, fish, vegetables and fruit. Too low a body concentration of MSM results in adverse physical and psychological stress, tissue and organ malfunction, fatigue and increased susceptibility to diseases.

Attempts have been made to design multivitamin supplements specifically for joint pain relief. Examples include U.S. Pat. No. 6,924,273 and U.S. Patent Publication No. 20050182022, which relate to formulation with glucosamine sulfate, hyaluronic acid and a gel or paste agent.

There is a need for safe and effective formulations that provide joint pain relief, especially for acute pain relief, when used for both short-term and long-term therapy and which can be administered orally.

Citation or identification of any document in this application is not an admission that such document is available as prior art to the present invention.

### SUMMARY OF THE INVENTION

The invention is based, in part, on the Applicants' discovery that a combination of dietary supplements, supplemented with acetaminophen for treatment of acute pain, may relieve joint pain and is useful for short-term and long-term therapy.

The invention is based upon a formulation which may comprise, consist essentially of or consist of glucosamine, hyaluronic acid and methylsulfonylmethane (“MSM”) for daily use and optionally acetaminophen for acute pain. The hyaluronic acid may also encompass salts, such as sodium hyaluronate.

The invention also provides for a caplet or tablet which may comprise a formulation which may comprise, consist essentially of or consist of glucosamine, hyaluronic acid and methylsulfonylmethane (“MSM”) for daily use and optionally acetaminophen for acute pain. The hyaluronic acid may also encompass salts, such as sodium hyaluronate.

Advantageously, dosage per caplet or tablet of glucosamine is about 180 mg to about 4 g, hyaluronic acid or a sodium salt thereof is about 1 mg to about 200 mg, methylsulfonylmethane (“MSM”) is about 100 mg to about 2 g. When included, the dosage of acetaminophen is about 100 mg to about 1 g, advantageously 500 mg.

The caplet or tablet may further comprise artificial colors, calcium carbonate salts, calcium phosphate salts, carboxymethylcellulose, carnauba wax, cellulose, corn starch, croscarmallose sodium, hydroxypropylcellulose, iron oxide, magnesium stearate, mannitol, methylcellulose, microcrystalline cellulose, polyethylene glycol, polyvinyl alcohol, polyvinylpolypyrillidone, polyvinylpyrillidone, silicon dioxide, sodium starch glycolate, sorbitol, stearic acid, sucralose, talc, titanic dioxide and xylitol or any combination thereof. Preferably, the caplet or tablet does not contain gluten, preservatives, sugar, sodium, milk, yeast, artificial colors, artificial flavors or any combination thereof.

Advantageously, the caplet or tablet may be enteric coated and may further comprise anionic polymers of methacrylic acid, artificial colors, calcium carbonate salts, calcium phosphate salts, carboxymethylcellulose, carnauba wax, cellulose, cellulose derivatives, corn starch, croscarmallose sodium, glycerol monostearate, hydroxypropylcellulose, iron oxide, magnesium stearate, mannitol, methacrylates, methyl vinyl ether derivatives, methylcellulose, methylphalates, microcrystalline cellulose, polyethylene glycol, polymethacrylates, polyvinyl alcohol, polyvinylpolypyrillidone, polyvinylpyrillidone, silicon dioxide, sodium acetate phalate, sodium starch glycolate, sorbitol, stearic acid, sucralose, talc, titanic dioxide and xylitol or any combination thereof.

In another advantageous embodiment, the caplet or tablet may be a controlled release of the active acetaminophen to allow up to 12 hours of pain relief. The controlled release caplet or caplet may comprise anionic polymers of methacrylic acid, artificial colors, calcium carbonate salts, calcium phosphate salts, carboxymethylcellulose, carnauba wax, cellulose, cellulose derivatives, corn starch, croscarmallose sodium, diglycerides, ethylcellulose, fatty acid esters, glycerol monostearate, gum (e.g., xanthan), hydroxypropylcellulose, iron oxide, magnesium stearate, mannitol, methacrylates, methyl vinyl ether derivatives, methylcellulose, methylphalates, microcrystalline cellulose, monoglycerides, polyethylene glycol, polymethacrylates, polyvinyl alcohol, polyvinylpolypyrillidone, polyvinylpyrillidone, silicon dioxide, sodium acetate phalate, sodium starch glycolate, sorbitol, stearex, stearic acid, sucralose, talc, titanic dioxide, wax (e.g., carnuba) and xylitol or any combination thereof.

The present invention also provides for method of providing joint pain relief which may comprise administering any of the above-described caplets or tablets which may comprise any one of the above-described formulations to a patient in need of joint pain relief. In an advantageous embodiment, the caplet or tablet may be administered once per day.

The invention further encompasses a kit for performing a method of providing joint pain relief which may comprise administering any of the above-described caplets or tablets which may comprise any one of the above-described formulations to a patient in need of joint pain relief and instructions for performing the method.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION

The invention is based, in part, on the Applicants' discovery that a combination of dietary supplements, supplemented with acetaminophen for treatment of acute pain, may relieve joint pain and is useful for short-term and long-term therapy.

The invention is based upon a formulation which may comprise, consist essentially of or consist of glucosamine, hyaluronic acid and methylsulfonylmethane (“MSM”) for daily use and optionally acetaminophen for acute pain. The hyaluronic acid may also encompass salts, such as sodium hyaluronate.

The acetaminophen formulations as described in U.S. Pat. Nos. 6,936,601; 6,926,907; 6,924,273; 6,916,788; 6,855,310; 6,852,336; 6,841,544; 6,833,362; 6,828,328; 6,787,164; 6,740,333; 6,702,850; 6,696,066; 6,686,390; 6,642,243; 6,627,234; 6,622,856; 6,613,346; 6,602,518; 6,593,331; 6,586,023; 6,569,439; 6,566,401; 6,566,361; 6,544,548; 6,528,097; 6,524,623; 6,511,982; 6,509,380; 6,492,334; 6,485,747; 6,482,831; 6,479,551; 6,475,526; 6,475,494; 6,458,809; 6,444,665; 6,440,983; 6,429,223; 6,413,512; 6,406,716; 6,391,886; 6,391,337; 6,391,294; 6,384,054; 6,383,527; 6,383,515; 6,375,957; 6,369,084; 6,369,082; 6,355,666; 6,350,467; 6,335,034; 6,309,669; 6,306,842; 6,303,632; 6,284,274; 6,277,384; 6,254,891; 6,245,802; 6,245,357; 6,242,493; 6,225,295; 6,221,377; 6,217,911; 6,217,907; 6,214,386; 6,187,338; 6,162,647; 6,159,500; 6,139,861; 6,127,352; 6,126,967; 6,077,533; 6,077,530; 6,057,347; 6,054,451; 6,048,540; 6,028,222; 6,007,841; 5,998,434; 5,972,916; 5,968,551; 5,965,167; 5,965,166; 5,945,416; 5,942,530; 5,919,826; 5,914,129; 5,897,880; 5,891,801; 5,891,477; 5,872,145; 5,863,922; 5,840,731; 5,837,729; 5,827,852; 5,776,462; 5,773,031; 5,739,139; 5,733,578; 5,724,957; 5,654,334; 5,639,475; 5,612,061; 5,603,959; 5,538,959; RE35,213; 5,474,757; 5,468,482; 5,466,865; 5,458,879; 5,417,980; 5,409,944; 5,409,709; 5,336,691; 5,322,689; 5,296,241; 5,273,759; 5,260,340; 5,238,686; 5,204,118; 5,154,926; 5,100,675; 5,036,097; 5,023,085; 5,011,688; 4,971,960; 4,971,785; 4,829,064; 4,822,781; 4,812,446; 4,794,112; 4,730,007; 4,703,045; 4,478,822; 4,476,115; 4,466,960; 4,460,368; 4,401,665; 4,314,989; 4,307,073; 4,260,629; 4,242,353; 4,237,140; 4,234,601; 4,233,317; 4,233,316; 4,233,315; 4,233,314; 4,233,313; 4,207,340; 4,132,788 and 4,049,803, the disclosures of which are incorporated by reference in their entireties, may be used in the present invention.

Brands of acetaminophen which may be used in the formulation of the present invention include, but are not limited to, Aceta Elixir, Aceta Tablets, Acetaminophen Uniserts, Actamin, Actamin Extra, Actamin Super2, Aminofen, Aminofen Max, Apacet Capsules, Apacet Elixir, Apacet Extra Strength Caplets, Apacet Extra Strength Tablets, Apacet, Infants', Apacet Regular Strength Tablets, Aspirin Free Anacin Maximum Strength Caplets, Aspirin Free Anacin Maximum Strength Gel Caplets, Aspirin Free Anacin Maximum Strength Tablets, Aspirin-Free Excedrin Caplets2, Banesin, Bayer Select Maximum Strength Headache Pain Relief Formula2, Dapa, Dapa X-S, Datril Extra-Strength, Equate, Excedrin, Feverall, Children's, Feverall, Infants', Feverall Junior Strength, Feverall Sprinkle Caps, Children's, Feverall Sprinkle Caps Junior Strength, Genapap Children's Elixir, Genapap Children's Tablets, Genapap Extra Strength Caplets, Genapap Extra Strength Tablets, Genapap, Infants', Genapap Regular Strength Tablets, Genebs Extra Strength Caplets, Genebs Regular Strength Tablets, Genebs X-Tra, Goody's, Liquiprin Children's Elixir, Liquiprin Infants' Drops, Neopap, Oraphen-PD, Panadol, Children's, Panadol, Infants', Panadol Junior Strength Caplets, Panadol Maximum Strength Caplets, Panadol Maximum Strength Tablets, Phenaphen Caplets, Redutemp, Snaplets-FR, St. Joseph Aspirin-Free Fever Reducer for Children, Suppap-20, Suppap-325, Suppap-650, Tapanol Extra Strength Caplets, Tapanol Extra Strength Tablets, Tempra, Tempra D.S, Tempra, Infants', Tempra Syrup, Tylenol Arthritis Extended Relief, Tylenol Children's Chewable Tablets, Tylenol Children's Elixir, Tylenol Children's Suspension Liquid, Tylenol Extra-Strength Adult Liquid Pain Reliever, Tylenol Extra Strength Caplets, Tylenol Extra Strength Gelcaps, Tylenol Extra Strength Tablets, Tylenol Infants' Drops, Tylenol Infants' Suspension Drops, Tylenol Junior Strength Caplets, Tylenol Junior Strength Chewable Tablets, Tylenol Regular Strength Caplets, Tylenol Regular Strength Tablets, Valorin and Valorin Extra in the United States and Abenol, Actimol Chewable Tablets, Actimol Children's Suspension, Actimol Infants' Suspension, Actimol Junior Strength Caplets, Anacin-3, Anacin-3 Extra Strength, Apo-Acetaminophen, Atasol Caplets, Atasol Drops, Atasol Forte Caplets, Atasol Forte Tablets, Atasol Oral Solution, Atasol Tablets, Excedrin Caplets2, Excedrin Extra Strength Caplets2, Exdol, Exdol Strong, Panadol, Panadol Extra Strength, Robigesic, Rounox, Tempra Caplets, Tempra Chewable Tablets, Tempra Drops, Tempra Syrup, Tylenol Caplets, Tylenol Children's Chewable Tablets, Tylenol Drops, Tylenol Elixir, Tylenol Gelcaps, Tylenol Junior Strength Caplets and Tylenol Tablets in Canada.

The glucosamine formulations as described in U.S. Pat. Nos. 6,984,667; 6,979,679; 3 6,979,470; 6,979,458; 6,974,841; 6,972,041; 6,946,551; 6,930,099; 6,924,273; 6,911,215; 6,908,630; 6,906,045; 6,902,739; 6,900,189; 6,887,497; 6,846,818; 6,841,544; 6,841,173; 6,838,451; 6,812,223; 6,797,289; 6,780,968; 6,767,899; 6,758,865; 6,716,458; 6,713,096; 6,709,682; 6,706,267; 6,689,399; 6,676,977; 6,660,308; 6,656,925; 6,653,294; 6,645,510; 6,645,482; 6,641,806; 6,635,625; 6,632,804; 6,632,449; 6,620,798; 6,608,041; 6,596,708; 6,596,699; 6,583,123; 6,579,544; 6,579,543; 6,541,045; 6,524,609; 6,506,785; 6,492,349; 6,486,307; 6,482,401; 6,479,469; 6,476,005; 6,455,309; 6,451,771; 6,447,809; 6,440,465; 6,432,929; 6,428,817; 6,423,347; 6,417,227; 6,402,783; 6,391,864; 6,346,519; 6,344,220; 6,333,304; 6,333,199; 6,291,527; 6,271,213; 6,267,786; 6,255,295; 6,231,608; 6,224,871; 6,165,983; 6,162,787; 6,159,954; 6,156,355; 6,149,946; 6,136,795; 6,117,851; 6,083,918; 6,046,179; 6,013,641; 5,984,858; 5,944,755; 5,939,403; 5,922,692; 5,916,565; 5,888,514; 5,849,336; 5,847,107; 5,843,919; 5,840,715; 5,804,594; 5,795,573; 5,733,572; 5,679,344; 5,587,363; 5,549,892; 5,364,845; 5,326,357; 5,217,962; 4,784,990; 4,772,591; 4,647,453; 4,325,962; 4,247,540; 4,146,721; 4,074,713; 4,074,366; 3,989,535 and 3,988,411, the disclosures of which are incorporated by reference in their entireties, may be used in the present invention.

Brands of glucosamine which may be used in the formulation of the present invention include, but are not limited to, Alacer, Country Life, Doctor's Best, Enzymatic Therapy, GNC, Inholtra, Natrol, Nature's Bounty, Nature's Way, Schiff, Swanson and Twinlab.

The hyaluronic acid formulations as described in U.S. Pat. Nos. 6,987,023; 6,986,995; 6,984,667; 6,974,862; 6,960,617; 6,953,784; 6,949,525; 6,949,251; 6,946,551; 6,939,562; 6,924,370; 6,924,273; 6,923,965; 6,911,436; 6,911,227; 6,902,584; 6,896,904; 6,893,466; 6,890,897; 6,886,568; 6,884,621; 6,884,428; 6,872,819; 6,869,938; 6,864,235; 6,852,696; 6,846,818; 6,821,747; 6,814,959; 6,812,220; 6,812,217; 6,806,259; 6,790,461; 6,790,455; 6,780,841; 6,777,000; 6,773,723; 6,765,069; 6,764,517; 6,761,887; 6,752,834; 6,737,072; 6,733,530; 6,723,709; 6,706,780; 6,703,444; 6,703,377; 6,703,041; 6,699,471; 6,692,435; 6,683,064; 6,667,296; 6,663,606; 6,654,120; 6,653,285; 6,652,887; 6,652,872; 6,645,945; 6,642,213; 6,638,538; 6,635,267; 6,630,486; 6,630,457; 6,626,950; 6,608,043; 6,608,041; 6,607,745; 6,602,294; 6,596,699; 6,585,987; 6,582,471; 6,582,228; 6,552,184; 6,541,460; 6,537,968; 6,533,820; 6,531,147; 6,518,401; 6,514,514; 6,509,322; 6,506,785; 6,503,539; 6,497,901; 6,495,127; 6,489,467; 6,482,231; 6,472,379; 6,468,308; 6,464,970; 6,451,301; 6,447,802; 6,444,447; 6,440,427; 6,432,929; 6,428,804; 6,417,173; 6,410,044; 6,391,861; 6,391,336; 6,387,413; 6,375,988; 6,372,257; 6,364,912; 6,335,035; 6,323,278; 6,312,720; 6,309,670; 6,303,585; 6,303,138; 6,294,202; 6,280,470; 6,258,870; 6,251,876; 6,251,436; 6,231,590; 6,224,635; 6,221,854; 6,218,373; 6,214,049; 6,204,254; 6,197,326; 6,194,392; 6,187,024; 6,183,737; 6,180,601; 6,159,955; 6,147,059; 6,147,054; 6,140,312; 6,136,793; 6,133,325; 6,129,761; 6,123,957; 6,114,314; 6,103,704; 6,099,952; 6,096,728; 6,087,344; 6,086,907; 6,069,135; 6,063,405; 6,060,053; 6,048,844; 6,042,610; 6,037,331; 6,031,017; 6,022,866; 6,020,326; 6,017,901; 6,017,900; 6,017,301; 6,010,692; 6,007,843; 5,990,381; 5,990,096; 5,990,095; 5,986,052; 5,985,851; 5,985,850; 5,981,825; 5,977,163; 5,977,088; 5,972,906; 5,972,385; 5,968,519; 5,962,433; 5,942,498; 5,942,245; 5,939,323; 5,932,560; 5,929,048; 5,925,626; 5,914,322; 5,914,314; 5,910,489; 5,906,997; 5,888,986; 5,888,984; 5,882,929; 5,882,664; 5,880,108; 5,874,500; 5,874,417; 5,872,094; 5,866,554; 5,866,165; 5,858,746; 5,852,002; 5,847,002; 5,843,743; 5,842,477; 5,834,274; 5,830,882; 5,830,504; 5,827,834; 5,824,658; 5,824,335; 5,817,644; 5,814,500; 5,811,410; 5,801,033; 5,800,541; 5,792,753; 5,776,193; 5,769,899; 5,767,106; 5,763,399; 5,753,266; 5,736,372; 5,735,903; 5,733,891; 5,733,572; 5,716,631; 5,713,959; 5,711,960; 5,709,883; 5,702,456; 5,690,961; 5,686,425; 5,681,353; 5,679,655; 5,674,857; 5,654,267; 5,645,592; 5,645,591; 5,644,049; 5,639,796; 5,639,738; 5,639,473; 5,635,207; 5,631,242; 5,631,241; 5,631,011; 5,624,915; 5,624,463; 5,622,707; 5,614,506; 5,612,321; 5,607,692; 5,591,724; 5,589,169; 5,585,361; 5,584,885; 5,583,120; 5,583,119; 5,583,118; 5,573,934; 5,550,112; 5,549,109; 5,529,987; 5,529,914; 5,520,916; 5,516,532; 5,510,418; 5,510,121; 5,503,848; 5,498,421; 5,476,666; 5,470,911; 5,460,832; 5,442,053; 5,425,766; 5,409,904; 5,403,592; 5,391,203; 5,386,013; 5,326,357; 5,324,775; 5,316,926; 5,306,311; 5,290,271; 5,268,165; 5,263,984; 5,258,043; 5,234,914; 5,219,360; 5,192,326; 5,180,808; 5,166,331; 5,140,016; 5,108,438; 5,095,037; 5,093,487; 5,080,924; 5,080,893; 5,079,236; 5,023,114; 5,015,677; 5,007,934; 4,979,959; 4,979,956; 4,973,493; 4,885,244; 4,880,429; 4,879,375; 4,808,576; 4,801,619; 4,795,741; 4,787,900; 4,784,991; 4,782,046; 4,780,414; 4,772,419; 4,749,570; 4,746,504; 4,735,902; 4,716,224; 4,272,522 and 4,141,973, the disclosures of which are incorporated by reference in their entireties, may be used in the present invention.

Brands of hyaluronic acid which may be used in the formulation of the present invention include, but are not limited to, GNC, Natrol and Nature's Way.

The methylsulfonylmethane (“MSM”) formulations as described in U.S. Pat. Nos. 6,924,273; 6,884,797; 6,841,173; 6,790,463; 6,733,751; 6,726,133; 6,653,352; 6,613,362; 6,589,555; 6,541,045; 6,444,234; 6,440,391; 6,417,227; 6,416,772; 6,328,987; 6,203,820; 5,569,679; 5,071,878; 4,973,605; 4,914,135; 4,863,748; 4,616,039; 4,568,547; 4,559,329; 4,514,421; 4,477,469; 4,296,130 and 4,227,013, the disclosures of which are incorporated by reference in their entireties, may be used in the present invention.

Brands of MSM which may be used in the formulation of the present invention include, but are not limited to, GNC, Natrol, Nature Made and Schiff.

In a particularly advantageous embodiment, the formulation may comprise, consist essentially of or consist of glucosamine, hyaluronic acid and methylsulfonylmethane (“MSM”) for daily use and optionally acetaminophen for acute pain.

The compounds of the present invention are advantageously useful in preventing or treating joint pain, such as, but not limited to, pain associated with inflammation of the joints, arthritis, osteoarthritis, and other degenerative joint diseases. Advantageously, the compounds of the present invention provides a method for joint pain relief which may comprise administering any of the above-described caplets or tablets which may comprise any one of the above-described formulations to a patient in need of joint pain relief. In an advantageous embodiment, the caplet or tablet may be administered once per day. Acetaminophen may be administered to treat acute pain relief. A patient may supplement the caplet or tablet of the present invention with acetaminophen when in need of acute joint pain relief, or alternatively, the patient may supplement the daily dose of the caplet or tablet with a weekly dose, for example, of acetaminophen. Any formulation of acetaminophen may be administered to supplement the formulations of the present invention.

When administered to a patient, a compound of the invention is preferably administered as component of a composition that optionally comprises a pharmaceutically acceptable vehicle The present compositions, which comprise a compound of the invention, are preferably administered orally. The compositions of the invention may also be administered by any other convenient route, for example, by infusion or bolus injection, by absorption through epithelial or mucocutaneous linings (e.g., oral mucosa, rectal, and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer the compounds of the invention.

In certain embodiments, the present compositions may comprise one or more compounds of the invention.

Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner. In most instances, administration will result in the release of a compound of the invention into the bloodstream.

In an advantageous embodiment, the administration is oral.

In an advantageous embodiment, the composition is enteric coated to prevent dissolution in the stomach. Advantageously, there is slow dissolution of the active substance until the tablet reaches the gastrointestinal tract. In a preferred embodiment, the compounds of the present invention are rapidly dispersed in the gastrointestinal tract.

In a less preferred embodiment, the compounds of the invention can be delivered in a vesicle, in particular a liposome (see Langer, 1990. Science 249:1527-1533; Treat et al, in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid).

In yet another less preferred embodiment, the compounds of the invention can be delivered in a controlled release system (see, e.g., Goodson, in Medical Applications of Controlled Release, supra, vol. 2, pp. 115-138 (1984)). Other controlled-release systems discussed in the review by Langer, 1990, Science 249:1527-1533) may be used. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of a target of a compound of the invention, thus requiring only a fraction of the systemic dose.

The present compositions can optionally comprise a suitable amount of a pharmaceutically acceptable vehicle so as to provide the form for proper administration to the patient.

In a specific embodiment, the tern “pharmaceutically acceptable” means approved by a regulatory agency of the Federal or a state government or listed in the U.S. Pharmacopeia or other generally recognized pharmacopeia for use in animals, mammals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening. lubricating and coloring agents may be used. When administered to a patient, the pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in Remington's Pharmaceutical Sciences, Alfonso R. Gennaro ed., Mack Publishing Co. Easton, Pa., 19th ed., 1995, pp. 1447 to 1676, incorporated herein by reference.

Advantageously, the composition is administered in the form of a caplet or a tablet. A tablet generally refers to a small solid pill containing a measured medicinal dose, usually intended to be taken orally. A caplet generally refers to a tablet of medicine taken orally. The terms tablet and caplet may be used interchangeably. The tablet or caplet may contain a liquid gel, a rapid release or extended release version of any of the herein disclosed formulations.

In a preferred embodiment, the compounds of the invention are formulated in accordance with routine procedures as a pharmaceutical composition adapted for oral administration to human beings. Compositions for oral delivery may be in the form of tablets, lozenges, aqueous or oily suspensions, granules, powders, emulsions, capsules, syrups, or elixirs, for example. Orally administered compositions may contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. Typically, compositions for intravenous administration comprise sterile isotonic aqueous buffer. Where necessary, the compositions may also include a solubilizing agent.

The amount of a compound of the invention that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed will also depend on the route of administration, and the seriousness of the disease or disorder, and should be decided according to the judgment of the practitioner and each patient's circumstances. However, suitable dosage ranges for oral administration are generally about 0.001 milligram to about 200 milligrams of a compound of the invention or a pharmaceutically acceptable salt thereof per kilogram body weight per day. In specific preferred embodiments of the invention, the oral dose is about 0.01 milligram to about 100 milligrams per kilogram body weight per day, more preferably about 0.1 milligram to about 75 milligrams per kilogram body weight per day, more preferably about 0.5 milligram to 5 milligrams per kilogram body weight per day. The dosage amounts described herein refer to total amounts administered; that is, if more than one compound of the invention is administered, or if a compound of the invention is administered with a therapeutic agent, then the preferred dosages correspond to the total amount administered. Oral compositions preferably contain about 10% to about 95% active ingredient by weight.

The dosage of glucosamine may be about 50 mg, about 75 mg, about 100 mg, about 125 mg, about 150 mg, about 200 mg, about 225 mg, about 250 mg, about 275 mg, about 300 mg, about 325 mg, about 350 mg, advantageously about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, about 5000 mg, about 5100 mg, about 5200 mg, about 5300 mg, about 5400 mg, about 5500 mg, about 5600 mg, about 5700 mg, about 5800 mg, about 5900 mg, about 6000 mg.

The dosage of hyaluronic acid or a sodium salt thereof may be about 0.1 mg, 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, advantageously about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 21 mg, about 22 mg, about 23 mg, about 24 mg, about 25 mg, about 26 mg, about 27 mg, about 28 mg, about 29 mg, about 30 mg, about 35 mg, 40 mg, about 45 mg, about 50 mg, about 55 mg, about 60 mg, about 65 mg, about 70 mg, about 75 mg, about 80 mg, about 85 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg or about 300 mg.

The dosage of methylsulfonylmethane may be about 50 mg, about 75 mg, about 100 mg, about 125 mg, about 150 mg, about 200 mg, about 225 mg, about 250 mg, about 275 mg, about 300 mg, about 325 mg, about 350 mg, advantageously about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, about 5000 mg, about 5100 mg, about 5200 mg, about 5300 mg, about 5400 mg, about 5500 mg, about 5600 mg, about 5700 mg, about 5800 mg, about 5900 mg, about 6000 mg.

The dosage of acetaminophen may be 50 mg, about 75 mg, about 100 mg, about 125 mg, about 150 mg, about 200 mg, about 225 mg, about 250 mg, about 275 mg, about 300 mg, about 325 mg, about 350 mg, advantageously about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg or about 4000 mg.

The caplet or tablet may further comprise artificial colors, calcium carbonate salts, calcium phosphate salts, carboxymethylcellulose, carnauba wax, cellulose, corn starch, croscarmallose sodium, hydroxypropylcellulose, iron oxide, magnesium stearate, mannitol, methylcellulose, microcrystalline cellulose, polyethylene glycol, polyvinyl alcohol, polyvinylpolypyrillidone, polyvinylpyrillidone, silicon dioxide, sodium starch glycolate, sorbitol, stearic acid, sucralose, talc, titanic dioxide and xylitol or any combination thereof. Preferably, the caplet or tablet does not contain gluten, preservatives, sugar, sodium, milk, yeast, artificial colors, artificial flavors or any combination thereof.

Advantageously, the caplet or tablet may be enteric coated and may further comprise anionic polymers of methacrylic acid, artificial colors, calcium carbonate salts, calcium phosphate salts, carboxymethylcellulose, carnauba wax, cellulose, cellulose derivatives, corn starch, croscarmallose sodium, glycerol monostearate, hydroxypropylcellulose, iron oxide, magnesium stearate, mannitol, methacrylates, methyl vinyl ether derivatives, methylcellulose, methylphalates, microcrystalline cellulose, polyethylene glycol, polymethacrylates, polyvinyl alcohol, polyvinylpolypyrillidone, polyvinylpyrillidone, silicon dioxide, sodium acetate phalate, sodium starch glycolate, sorbitol, stearic acid, sucralose, talc, titanic dioxide and xylitol or any combination thereof.

In another advantageous embodiment, the caplet or tablet may be a controlled release of the active acetaminophen to allow up to 12 hours of pain relief. The controlled release caplet or caplet may comprise anionic polymers of methacrylic acid, artificial colors, calcium carbonate salts, calcium phosphate salts, carboxymethylcellulose, carnauba wax, cellulose, cellulose derivatives, corn starch, croscarmallose sodium, diglycerides, ethylcellulose, fatty acid esters, glycerol monostearate, gum (e.g., xanthan), hydroxypropylcellulose, iron oxide, magnesium stearate, mannitol, methacrylates, methyl vinyl ether derivatives, methylcellulose, methylphalates, microcrystalline cellulose, monoglycerides, polyethylene glycol, polymethacrylates, polyvinyl alcohol, polyvinylpolypyrillidone, polyvinylpyrillidone, silicon dioxide, sodium acetate phalate, sodium starch glycolate, sorbitol, stearex, stearic acid, sucralose, talc, titanic dioxide, wax (e.g., carnuba) and xylitol or any combination thereof.

The invention also provides pharmaceutical packs or kits comprising one or more vessels containing one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound of the invention. In another embodiment, the kit comprises a therapeutic agent and a compound of the invention.

Advantageously, the invention provides kits for relieving joint pain comprising the caplets or tablets described herein, acetaminophen and instructions for daily administration of the caplet or tablet that may comprise, consist essentially of or consist of glucosamine, hyaluronic acid and methylsulfonylmethane (“MSM”) and administration of acetaminophen for acute pain. In one embodiment, the administration of acetaminophen is daily for 10 days (e.g., up to 4 g per day). In another embodiment, the administration of acetaminophen is weekly dose (e.g., up to 4 g per day), with a 28 g maximum weekly dose.

The invention also provides for kits for relieving joint pain comprising the caplets or tablets described herein instructions for daily administration of the caplet or tablet that may comprise, consist essentially of or consist of glucosamine, hyaluronic acid and methylsulfonylmethane (“MSM”) and administration of the caplet or tablet that may comprise, consist essentially of or consist of glucosamine, hyaluronic acid, methylsulfonylmethane (“MSM”) and acetaminophen for acute pain with a 4 g maximum daily dose for up to 10 days maximum. Undesirable toxicities of chronic acetaminophen can be avoided by limiting the daily dose to less than 4 g during short-term therapy (e.g., up to 10 days) and less than 2.6 g daily during chronic therapy.

The compounds of the invention are preferably assayed in vitro and in vivo, for the desired therapeutic or prophylactic activity, prior to use in humans. For example, in vitro assays can be used to determine whether it is preferable to administer a compound of the invention alone or in combination with another compound of the invention and/or a therapeutic agent. Animal model systems can be used to demonstrate safety and efficacy.

Other methods will be known to the skilled artisan and are within the scope of the invention.

In certain embodiments of the present invention, a compound of the invention can be used in combination therapy with at least one other therapeutic agent. The compound of the invention and the therapeutic agent can act additively or, more preferably, synergistically. In a preferred embodiment, a composition comprising a compound of the invention is administered concurrently with the administration of another therapeutic agent, which can be part of the same composition as or in a different composition from that comprising the compound of the invention. In another embodiment, a composition comprising a compound of the invention is administered prior or subsequent to administration of another therapeutic agent. As many of the disorders for which the compounds of the invention are useful in treating are chronic, in one embodiment combination therapy involves alternating between administering a composition comprising a compound of the invention and a composition comprising another therapeutic agent, e.g., to minimize the toxicity associated with a particular drug. The duration of administration of the compound of the invention or therapeutic agent can be, e.g., one month, three months, six months, a year, or for more extended periods. In certain embodiments, when a compound of the invention is administered concurrently with another therapeutic agent that potentially produces adverse side effects including, but not limited to, toxicity, the therapeutic agent can advantageously be administered at a dose that falls below the threshold at which the adverse side is elicited.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A caplet or tablet comprising a formulation consisting essentially of glucosamine, hyaluronic acid and methylsulfonylmethane, wherein a dosage per caplet or tablet of glucosamine is about 180 mg to about 4 g, hyaluronic acid or a sodium salt thereof is about 1 mg to about 200 mg and methylsulfonylmethane (“MSM”) is about 100 mg to about 2 g.

2. The caplet or tablet of claim 1 further comprising an artificial color, calcium carbonate salt, calcium phosphate salt, carboxymethylcellulose, carnauba wax, cellulose, corn starch, croscarmallose sodium, hydroxypropylcellulose, iron oxide, magnesium stearate, mannitol, methylcellulose, microcrystalline cellulose, polyethylene glycol, polyvinyl alcohol, polyvinylpolypyrillidone, polyvinylpyrillidone, silicon dioxide, sodium starch glycolate, sorbitol, stearic acid, sucralose, talc, titanic dioxide and xylitol or any combination thereof.

3. The caplet or tablet of claim 1 wherein the caplet or tablet does not contain gluten, preservatives, sugar, sodium, milk, yeast, artificial colors, artificial flavors or any combination thereof.

4. The caplet or tablet of claim 1 wherein the caplet or tablet is enteric coated.

5. A caplet or tablet comprising a formulation consisting essentially of glucosamine, hyaluronic acid, methylsulfonylmethane and acetaminophen.

6. The caplet or tablet of claim 5 wherein a dosage per caplet or tablet of glucosamine is about 180 mg to about 4 g, hyaluronic acid or a sodium salt thereof is about 1 mg to about 200 mg and methylsulfonylmethane (“MSM”) is about 100 mg to about 2 g and acetaminophen is about 100 mg to about 1 g.

7. The caplet or tablet of claim 5 further comprising artificial color, calcium carbonate salt, calcium phosphate salt, carboxymethylcellulose, carnauba wax, cellulose, corn starch, croscarmallose sodium, hydroxypropylcellulose, iron oxide, magnesium stearate, mannitol, methylcellulose, microcrystalline cellulose, polyethylene glycol, polyvinyl alcohol, polyvinylpolypyrillidone, polyvinylpyrillidone, silicon dioxide, sodium starch glycolate, sorbitol, stearic acid, sucralose, talc, titanic dioxide and xylitol or any combination thereof.

8. The caplet or tablet of claim 5 wherein the caplet or tablet does not contain gluten, preservatives, sugar, sodium, milk, yeast, artificial colors, artificial flavors or any combination thereof.

9. The caplet or tablet of claim 5 wherein the caplet or tablet is enteric coated.

10. A method of relieving joint pain comprising administering the caplet or tablet of claim 1 to a patient in need of joint pain relief.

11. The method of claim 10 wherein the caplet or tablet of claim 1 is administered once per day.

12. A method of relieving joint pain comprising administering the caplet or tablet of claim 5 to a patient in need of joint pain relief.

13. The method of claim 10 wherein the caplet or tablet of claim 1 is administered once per day.

14. A kit for performing the method of claim 10 comprising the caplet or tablet of claim 1, acetaminophen and instructions for daily administration of the caplet or tablet of claim 1 and administration of acetaminophen for acute pain.

15. The kit of claim 14, wherein the administration of acetaminophen is a maximum of about 4 g per day for a maximum of ten days.

16. A kit for performing the method of claim 10 comprising the caplet or tablet of claim 1, the caplet or tablet of claim 5 and instructions for daily administration of the caplet or tablet of claim 1 and administration of the caplet or tablet of claim 5 for acute pain.

17. The kit of claim 16, wherein the administration of the caplet or tablet of claim 5 is is a maximum of about 4 g of acetaminophen per day for a maximum of ten days.

18. The caplet or tablet of claim 1, wherein the caplet or tablet is a liquid gel caplet or tablet, a rapid release caplet or tablet or an extended release caplet or tablet.

19. The caplet or tablet of claim 5, wherein the caplet or tablet is a liquid gel caplet or tablet, a rapid release caplet or tablet or an extended release caplet or tablet.
